(19) United States
(12) Patent Application Publication
(10) Pub. No.: US 2020/0197653 A1
(43) Pub. Date: Jun. 25, 2020

(54) MULTI-MODAL PAIN MANAGEMENT DEVICE

(71) Applicant: Tswelopele Labs, Inc., Midlothian, VA (US)

(72) Inventors: Andre P. Boezaart, Gainesville, FL (US); Stephen O. Vose, Gainesville, FL (US); Timothy J. Bechen, Midlothian, VA (US)

(21) Appl. No.: 16/720,469

(22) Filed: Dec. 19, 2019

Related U.S. Application Data
(60) Provisional application No. 62/783,294, filed on Dec. 21, 2018.

(51) Int. Cl. A61M 19/00; A61F 5/37; A61N 1/36

(52) U.S. Cl. CPC A61M 19/00; A61F 5/37; A61N 1/36021; A61M 2205/054; A61M 2025/1079; A61M 2230/06; A61M 2230/63; A61M 2209/088; A61M 2205/3306; A61M 2205/502

(57) ABSTRACT

A mobile pain management device for managing pain of a patient, the device includes a nerve block engageable with a first nerve of the patient and an electrical stimulation device engageable with a second nerve of the patient. The device includes an analgesic dispensing system having analgesic solution stored therein, the analgesic dispensing system connected to the nerve block such that when the nerve block is activated, the dispensing system provides at least a portion of the analgesic solution to the nerve block. The device includes a control module, in response to executable instructions, providing pain relieve the patient through engagement of the nerve block and the electrical stimulation. And the device includes a sensor detecting feedback from the patient such that the control module modifies the providing of pain relief to the patient based on the feedback.

### RELATED APPLICATIONS

The present application relates to and claims priority to U.S. Provisional Patent Application Ser. No. 62/783,294 filed Dec. 21, 2018.

### COPYRIGHT NOTICE

A portion of the disclosure of this patent document contains material that is subject to copyright protection. The copyright owner has no objection to the facsimile reproduction by anyone of the patent document or the patent disclosure as it appears in the Patent and Trademark Office patent files or records, but otherwise reserves all copyright rights whatsoever.

### FIELD OF INVENTION

The present invention relates generally to mobile pain management and more specifically to a mobile device including multiple modalities of pain management with improved user feedback and connectivity.

### BACKGROUND

With increasing medical costs, cost saving measures are in high demand. There is always the attendant trade-off between patient safety with minimizing costs. Hospitalization is a significant cost and thus reducing in-patient times directly lowers medical expenses.

For many surgical procedures, overnight in-patient stays are primarily directed towards pain management. There are numerous concerns in allowing indirect supervision for a patient under a pain-management plan. Concerns can include too much pain medication, leading to reduced inactivity or even possible death. Concerns also include pain management dependency, including possible addiction. Problems also arise with patients failing to take medication, taking improper dosages, or even mis-timing dosing.

Concurrently, there is an increase trend in ambulatory pain management techniques. Ambulatory continuous peripheral nerve blocks (ACPNB) are becoming increasing popular to provide safe, high-quality post-operative analgesia for outpatient surgery, reducing attendant medical costs.

As patients presenting for outpatient surgery become increasingly aged and complex, technology available to treat post-operative pain must concurrently advance to meet this need. The concept of treating terminal cancer pain with ACPNB is growing exponentially, with a sub-specialty of acute pain management growing with anesthesiology.

An existing study shows the feasibility for mobile pain management. This is according to the article “Internet remote control of pump settings for postoperative continuous peripheral nerve blocks: A feasibility study in 59 patients” by P. Macaire et al., published in Société Français d'Anesthésie et de Réanimation in 2013. In this feasibility study, 59 patients were given post-operative pain management using remote-control medicine-dispensing devices. Patients answered 10 specific questions about pain management directly on the device. If any of the answers were outside preset ranges, an anesthesiologist received a text message, could then log into a dedicated website, and electronically adjust the dispension flow rate.

This feasibility study was performed completely within the surgery ward of the hospital and relied exclusively on the patient providing a numbered 1-10 feedback answer to preset questions. This study did not actually provide ambulatory pain management and used rudimentary feedback to preset questions. The study supports feasibility of ambulatory pain management in general, but fails to include many features for patient comfort, security of medicine management, multiple modes of pain management, and varying means for collecting and analyzing patient data.

Providing proper pain management for surgical ambulatory patients must be safe and effective. While techniques and skills of many clinicians to safely and effectively place continuous nerve blocks are reaching maturity, there is limited pump technology for ACPNB infusion. Existing products used for ambulatory infusion pumps (AIPs) consist of mainly two types: elastomeric devices and electrical pumps.

Elastomeric pumps, such TeleFlex AutoFusor available from Teleflex Medical is a simple elastic reservoir filled with local anesthetic medication. A restrictor in the outflow tubing governs the infusion rate such that as the volume of medication reduces, so does the flow rate. This pump fails to provide for physician control beyond the initial setting.

Electrical pumps, such as the ambIT® pump available from Summit Medical Products, improves the accuracy in the volume of delivered medication and allows a bolus function.

But both pump types suffer from several complications preventing ambulatory care. First off, these pumps require the patient to carry a large bag of medication, typically in a fanny pack or some other cumbersome carrying case. Also, both pump types lack remote interfacing functionality, thus not individually suitable for outpatient or ambulatory care.

U.S. Pat. No. 8,551,038, (the '038 Patent), describes a pump infusion system allowing for ambulatory care as the pump can be remotely programmed. While allowing for ambulatory care, the '038 Patent limits interactivity and remote patient monitoring. The '038 Patent presents questions for the patient on a display screen, the patient feedback being a numbered response. The '038 Patent discloses acquiring additional feedback using an implanted electrode or sensor(s) in the catheter tip. Using a wireless connection, the '038 Patent then allows a medical practitioner to modify the dispense rate in response to the feedback.

The '038 Patent is narrowly-tailored in not only user interfacing, data collection, but also pain management options. The '038 Patent limits the patient feedback to set of questions on a small display screen on the device itself. The '038 Patents notes a limited selection of narrowly-tailored supplemental sensors. Moreover, the '038 Patent is narrowly tailored to drug dispensing in a trial setting, whereby the on-screen feedback and sensor feedback provide for clinical trial information, instead of a direct focus on patient pain management.

Another pain management technique is the use of electrical stimulation of a nerve. Existing techniques are known for using varying waveforms for nerve stimulation. These known techniques, including for example as described in U.S. Pat. No. 8,275,461, allow for pain management by nerve stimulation.

Thus, there are currently multiple modalities for pain management. Each modality has a timing and purpose but existing technology fails to integrate these modalities into a common unit. Therefore, there exists a need for a mobile pain management device utilizing multiple pain management modalities with improved patient feedback, reducing medication dependencies.

### BRIEF DESCRIPTION

The present invention provides for a multi-modal mobile pain management device for managing pain management, the device is also referred to as a smart anesthetic infusion device (SAID). The present invention enables post-operative pain management reducing or eliminating unnecessary post-operative hospitalization(s).

The SAID includes a nerve block engageable with a first nerve of a patient. The nerve block can be a peripheral continuous nerve block. The nerve block offers the first pain management modality.

The SAID includes an electrical stimulation device engageable with a second nerve of the patient. Depending on the pain management objective, the first nerve and the second nerve of the patient may be the same nerve, but could also be different nerves. The electrical stimulation device offers a second pain management modality.

The SAID further includes an analgesic dispensing system having analgesic solution stored therein. The analgesic solution can be any suitable analgesic, including opioid or non-opioid solutions. The dispensing system is connected to the nerve block such that upon nerve block activation, the dispensing system provides at least a portion of the analgesic solution to the nerve block.

In one embodiment, the analgesic dispensing system may include a second container of mixing solution, such as saline, for combination with the analgesic solution prior to dispensing to the nerve block. In this embodiment, the dispensing system can regulate or modify the potency of the analgesic being dispensed by the nerve block.

The SAID further includes a control module that controls the operations of the different pain management modalities. The control module receives feedback information from one or more sensors associated with the patient. The sensors can detect pain levels, such as for example a motion sensor detecting if the patient is active or a biometric sensor registering elevated heart rate or blood pressure. The control module can then manage pain management using the multiple modalities based on the feedback data from the sensors.

In further embodiments, the control module includes a wireless communicator, a control processor, and user interface component(s) such as a screen, a speaker, microphone, etc. The wireless communicator communicates with both local, near-field, devices as well as networked devices. While the control module can interface with the user via a local user interface, the control module primarily operates via a wireless connection to a mobile device running an application. Thus, user interface functionality on the control module may be minimized.

It is well recognized, pain management is not directed exclusively to total pain mitigation. As part of the healing process, the patient should retain feeling and limb sensation. The objective is not to completely eliminate pain but rather maintain it as a comfortable level so as to participate in his or her own recovery.

In one embodiment, the SAID can alternate pain management techniques switching from analgesic nerve block to electrical stimulation. For example, the patient in a post-operative environment may utilize analgesic dispensing via the nerve block for the first 24 or 48 hours. Electrical stimulation can be used for pain management as part of the medicinal weening process, but allowing for maintaining pain management for the patient.

Via the control module, the dosage of the analgesic can be reduced by downwardly regulating the amount of pain medicine, such as increasing a mixing amount of the mixing liquid. Then, after a period of time, the control module can change the pain management modality from drug-based to electrical stimulation.

In another embodiment, the SAID includes a mobile or external communication system. The device, via the communication system, provides for communicating not only with local-based sensors, but also with networked systems. For example, the device can communicate with a hospital or medical team providing timely feedback for pain management operations. The communication may be across a networked connection, e.g. Internet, connections via wireless network, via mobile network, etc.

In one embodiment, the SAID may be directly affixed to a limb that was subject to the operation. The analgesic system may be directly associated with device or can be connected via dispensing tubes, for example disposed in a container resembling a hip flask affixable to a waistband or belt.

Thereby, the present invention provides for improved pain management techniques in a mobile application reducing reliance on post-operative hospitalizations for pain management purposes, as well as reducing reliance on addictive medications, such as opioids.

The user interface device is a handheld or otherwise portable computing device in communication with the control module. For example, the user interface device can be a mobile phone or table computer running an application (e.g. an “app”) that communicates with the control module. The application further includes user interactivity for engaging with the user or a caretaker assisting with the user. For example, user interface functionality may include asking question of the user or providing reminders for engaging in movement. The interface can also include requests to increase or decrease medication, call for medical assistance, etc.

The SAID further employs ancillary connected devices for monitoring user movement. Ancillary devices may be any device or devices that assist in detecting movement or otherwise helps monitor the user. For example, a foot pod device can attach to a shoe or sock, wirelessly communicating with the control module, directly or via the user interface device, to detect movement. Another example can be motion sensors placed above and below the knee of the user to detect if the user is moving the leg and flexing the knee joint. Other ancillary devices can include any number of different options, including for example using a GPS position tracker within a mobile phone, a smart watch, a camera to detect facial expressions, etc.

The SAID further operates in coordination with a network processing device. The network processing device connects the user with medical supervision. Via the network processing device, medical supervision can monitor the rate of medication dispensing, responses to user feedback questions, answer user questions in real-time, detect or monitor user movement, monitor user pain management status, etc. In further embodiments, the medical supervision may not actively monitor, but rather be immediately notified if the user's readings fall outside of any a preset range, for example detecting that a user's self-reporting pain level is too high or a medication dispensing rate has been increased by a certain percentage over a certain time range.

The combination of these various elements therein allows for ambulatory monitoring of pain management. By harnessing existing mobile movement technology, the present method and system reduces unnecessary hospitalizations relating solely for pain management. Moreover, the SAID provides multi-modal pain management via anesthetic and neuromodulation.

### BRIEF DESCRIPTION OF THE FIGURE

FIG. 1 illustrates one embodiment of a mobile pain management device;

FIG. 2 illustrates a block diagram of the operating environment of the mobile pain management device of FIG. 1;

FIG. 3 illustrates block diagram of one embodiment of a control module of the pain management device;

FIG. 4 illustrates one embodiment of a peripheral continuous nerve block device of the mobile pain management device of FIG. 1;

FIG. 5 illustrates a block diagram of one embodiment of a continuous nerve block medication distribution module operating with the nerve block device of FIG. 4;

FIG. 6 illustrates one embodiment of an electrical neuromodulation device of the mobile pain management device of FIG. 1;

FIG. 7 illustrates a flowchart of one embodiment of a multi-modal ambulatory pain management method; and

FIGS. 8-9 illustrate one embodiment of the mobile pain management device in operation with a limb immobilization device.

### DETAILED DESCRIPTION

The present invention provides for pain management and mitigation via a SAID operating in conjunction with wireless and mobile technologies.

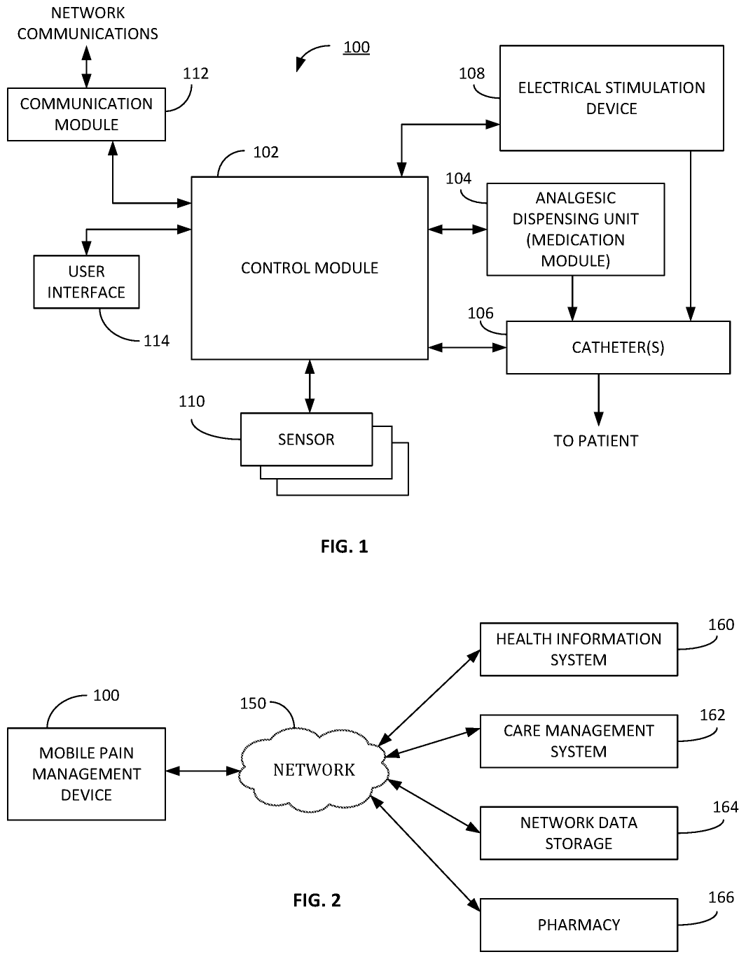

FIG. 1 illustrates one embodiment of a SAID 100 including a control module 102, an analgesic dispensing unit 104, a catheter 106, an element stimulation device 108, a plurality of connected sensors 110, a communication module 112, and at least one user interface 114.

The control module 102 includes processing capabilities for controlling the medication module 104, as well as neuromodulation controller 108, processing data from sensors 110, communicating to external devices or systems via the communication module 112, as well engaging with the user interface 114. The control module 102 processing operations are performed by at least one of hardware and/or software executing on hardware, the hardware including one or processing devices and memory device(s).

The control module 102 communicates with the analgesic dispensing system 104. The medication is dispensed to the patient via the catheter 106, such as a peripheral continuous nerve block catheter as recognized by one skilled in the art. In one embodiment, the catheter may be a catheter similar to U.S. Pat. No. 9,168,351 titled “Instrument for continuous discharge of anesthetic drug.” In another embodiment, the catheter may be similar to U.S. Pat. Nos. 6,456,874, 6,973,346, and/or 7,386,341.

The control module 102 further communicates with the electrical stimulation device 108 for controlling neuromodulation to the patient via the catheter 106 or via a separate catheter (not expressly shown). As described in greater detail below, the SAID 100 provides for multi-modal pain management, allowing for the control module 102 to change the pain management for the patient between analgesic and electrical stimulation. The electrical stimulation device 108 may provide electrical stimulation to the patient using known stimulation techniques, including for example as described in U.S. Pat. No. 8,275,461 entitled “Pain relieving waveform system and method.”

The control module 102 further interacts with one or more of the sensors 110. The sensors 110 may be any suitable type of sensor. The sensors 110 can be any electronically connected sensors, wired or wirelessly connected, communicating information with the control module 102. By way of example, the sensor 110 can be a smart watch or motion-tracking, e.g. GPS enabled, watch or device. The sensor 110 may be a foot pod connectable to a shoe to track movement or communicate movement. The sensor 110 may be one or more movement-tracking devices that can be attached to the patient's body to detect limb movement. The sensor 110 may include a heart rate monitor or any other biofeedback device, such as an oximeter device. The sensors 110 may also be embedded or operational within externally connected devices, such as for example a mobile phone, tablet or other computing device. In another embodiment, the sensor 110 may be a microphone or camera for capturing audio or video, respectively.

The control module 102 wirelessly communicates with external device(s) via the communication module 112. By way of example, the control module 102 may communicate with a mobile device, including for example, but not limited to, a smart phone, a table computer, a mobile computer, a function-specific handheld device, an electronic assistance system, or a smart watch. The mobile device may include an application running thereon, the application including interface functionality for communicating between the patient or a caretaker, the control module 102, and the communication module 112.

In another embodiment, the communication module 112 provides for network connectivity, such as the Internet, a private network, a combination of networks including a mobile communication network connecting to other networks, etc. The network connectivity operates in accordance with known communication techniques, including such as but not limited to wi-fi connectivity to network(s) using known protocols, or connecting via a mobile or cellular connection point. In one embodiment, the control module 102 operates in a home environment connecting via a home wi-fi network, but the module 102 may also operate outside of a wi-fi network using a cellular or other type of network connectivity allowing for maintaining operative communication with the network via communication module 112.

The communication module 112 allows for one or more processing devices disposed on the network for communicating with and tracking information from the control module 102. Networked controllers can not only monitor the activities of the patient via the control module 102, but also communicate with additional resources, e.g. doctors, pharmacy, insurance company, etc. The networked controller allows for operation of the control module 102, but serves as a back-up to insure proper pain management and patient-safety. For example, if the control module 102 detects abnormal patient behavior or medication dispensing, the control module 102 can notify the networked controller to engage a doctor or medical team member.

The SAID 100 provides for multi-modal pain management for a patient with the control module 102 operative to switch between pain control via analgesic from the dispensing until 104 and electrical stimulation from the electric stimulation device 108. The control module 102 can respond to patient feedback via the sensor(s) 110 and modify pain management controls accordingly. Similarly, the module 102 may alter or pause pain management controls for rehabilitation or other recovery purposes, as controlled by the patient, a medical profession, a therapist, or other person. For example, physical therapy may require the patient to have proprioceptive awareness, including to insure against falling, so the analgesic dispensing unit 104 can be turned off or dosage greatly reduced ahead of the physical therapy session.

The user interface 114 may be any suitable devices or devices allowing for user input and output. In one embodiment, the user interface 114 may be a smartphone, tablet computer, dedicated mobile processing device, or any other suitable computing-based input/output device. For example, the user interface 114 can be an output device displaying information associated with the control module 102. For example, an application or other software running on the user interface 114 can including display of update messages regarding the received user feedback, as well as changes to the pain control functions.

One embodiment may include a display screen allowing for a patient or person assisting the patient to manually enter data into the user interface 114, such as via a touchscreen or other suitable interface. The touchscreen can allow for text-based entry or greater interactivity beyond merely selecting a number between 1-10.

In one embodiment, the control module 102 may receive instructions to reduce the amount of pain medicine being dispensed to the patient. By way of example, the control module 102 may reduce analgesic dispensing because the patient has an upcoming physical therapy session, the patient has indicated the pain levels have gone down, the reduction is part of a standard weening process, the sensors detect the patient has been immobile for too long of a period of time, or any other suitable reason. The user interface 114 can act as an output device to display an update message notifying the patient that the analgesic dispensing is being adjusted. Similarly, if the analgesic rate is to be increased, the output display can indicate that the rate is being increased.

The user interface 114 can be dedicated screen with the SAID or in another embodiment can be a separate device, such as in wireless commination, such as a mobile phone, computer, tablet, in wireless communication via the communication module 112.

In one embodiment, the adjustment of the analgesic flow rates are determined by external sources, such as a care management system described in greater detail below. Therefore, the output of the update message can be based on instructions from the care management system.

In addition to direct patient pain management, the SAID 100 is further operative for communication with external systems. FIG. 2 illustrates one embodiment of the SAID 100 communicating with multiple system via a network 150. With reference to FIG. 1, the SAID 100 communicates using the communications module 112.

In the example of FIG. 2, the SAID 100 communicates with any number of external systems, including for example a health information system 160, a care management system 162, a network data storage system 164, pharmacy system 166.

The health information system 160 can be a hospital information tracking system, such as managing post-operative care statistics. For example, a health care provider can track data for how long the patient uses the different pain management modalities, track how long before the patient reduces dependency on analgesic or other anesthetics, other any other suitable tracking data. By way of example, a hospital may track patient information for billing and record keeping purposes. The health information system 160 can monitor patient records relating to activities with the control module 102 of FIG. 1, such as recording answer to questions, tracking movements, tracking amount of pain medication intake, tracking prescription refills, requests for doctor consultations, etc.

The care management system 162 can be associated with nurses, doctors, or other health care professionals. This can help monitor feedback from sensors, detect when the patient is experience high pain thresholds or remaining inactive for extended period of time. The system 162 can also remotely monitor the patient, for example using sensors to see real-time patient data, including for example a camera to view the patient, heart rate data, mobility tracking data, blood pressure data, etc. The system 162 can include tracking the patient vitals and other measurements, performing diagnostics using patient and/or sensor measurements, intervening if requested or directed to be the control module, etc.

The network data storage 164 can be an online data storage platform for monitoring all patient information. For example, an insurance company may track recovery data to be better monitored, authorizing post-operative SAID treatments over hospitalizations. The SAID 100 can upload data to the storage 164, including for example security encoding or removal of patient-identifying information, as appropriate. The data can be centrally stored, or in another embodiment may be stored in a distributed data environment using encoded technologies, such as a blockchain data storage process to ensure data integrity.

The pharmacy 166 may be a system associated with a drug delivery or refill service. For example, if the analgesic in the dispensing unit (104 of FIG. 1) is running out, a prescription refill may be automatically ordered.

Other control modules, not shown, may be any other suitable module or system as recognized by one skilled in the art. For example, one network may be a government or insurance network. The government may include a regulatory body, such as the Food and Drug Administration or the Drug Enforcement Agency tracking information relating to any controlled or scheduled substances being distributed and delivered via the medication module. A government database may include Medicaid or Medicare or Veterans Affairs Administration tracking device activity for patient advocacy purposes. Insurance database and systems may include processing information about the particular patient to monitor compliance or track efficacy of the device and/or for re-imbursement purposes, by way of example.

One or more sensors may further detect removal of an opioid container, including for tracking medication. For example, one embodiment may include a locking mechanism preventing removal of the opioid container without external authorization, such as a PIN or an electronic key generated and transmitted from an external source, similar to two-party authentication technologies for remote application and web-based login security protocols.

The above examples are exemplary in nature and not expressly limiting. It is recognized that any suitable external system may be in communication with the SAID 100.

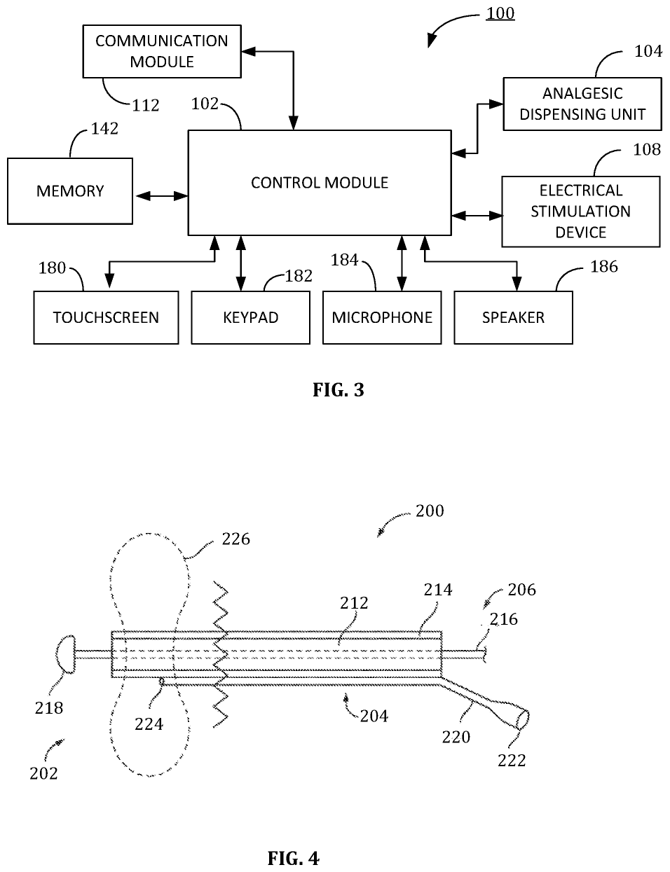

FIG. 3 illustrates a further embodiment of the SAID 100 with control module 102 and a memory 142 having executable instructions stored therein. The module 102 performs operations in response to the instructions, including operations as described herein.

The module 102 allows for user interaction via any number of user interface devices, including for example touchscreen 180, keypad 182, speaker 184, and microphone 186.

The module 102, in response to the executable instructions, operates to manage modalities of pain medication, the analgesic dispensing unit 104 and the electrical stimulation device 106, but also to monitor and engage with the patient via one or more user interface components, such as 180-186.

The control module 102 provides dosage and/or dispense rate signals to the analgesic dispensing unit 104. Based on this signal, proper medication is dispensed to the user for managing pain. Or, to avoid dependence on opioids or other medicines, the control module 102 can reduce or turn off the analgesic, or including switching to electrical stimulation instead.

The adjustment of the dosage instruction, turning off and activating electrical stimulation can be modified by the control module 102 in response to user feedback. For example, in one embodiment the control module 102 can ask questions for user feedback on the touchscreen 180 or via the keypad 182. One such question may be a pain level question, asking if about the patient's current pain level on a preset scale or a set of varying facial images. Based on entering a response, the control module 102 may then consider adjusting the dosage instructions, either automatically or via checking with a networked system as described above.

Other examples of user feedback can include the speaker 184 asking a question and the microphone 186 using voice capture technology. In one embodiment, the control module 102 may utilize autonomous assistance technology, e.g. “Alexa” from Amazon®, “Siri” from Apple®, “Google Home” from Google®, to conduct interactions with the patient.

Another example of user interfacing may include a camera embedded within the control module 102 or via a connected device for capturing a facial image of the patient. For example, a patient may have a home camera, home security system, or other device with a camera such as a laptop computer or a desktop computer. Image recognition software may analyze the image to detect if the patient is expressing pain, discomfort, or even tracking patient movement.

In addition to the expressly illustrated interface components 180-186, the control module 102 can utilize the communication module 112 to communicate with any number of externally connected devices. For example, multiple devices may utilize Bluetooth® technology or other wireless technology to communicate.

In one embodiment, the above-described camera may utilize a camera available within the mobile device communicating with the control module 102 via the transmitter/receiver 112.

Other wirelessly connected devices, such as but not limited to a smart watch, heart rate monitor, pulse-ox monitor, movement detection sensor, blood sugar sensor, can share information with the control module 102 via the communication module 112. For example, the heart rate monitor can periodically or continuously send heart rate information to the control module 102 allowing for tracking the patient's heart rate. If there is a jump in heart rate, that can indicate an increase in pain, whereas a reduction in heart rate can indicate too much pain medication.

In another example, if the wirelessly-connected device is a foot pod or movement sensor attached to the patient, this can monitor movement. In the example of pain management for post-operative care where the patient is required to be intermittently mobile, these devices can indicate if the patient has moved. Lack of movement could indicate improper pain management levels, such as not enough medicine and the patient is immobile because of pain or too much medicine and the patient is not able to move.

Where the control module 102 communicates with the analgesic dispensing unit 104 to dispense medication and communicates with the directly and wirelessly connected interfacing devices to monitor the patient, the module 102 further communicates with the networked controller(s) for external tracking and monitoring of the patient. The networked controller(s) allow for greater medical supervision in an as-needed scenario. Various embodiments provide for notifications to the networked controller(s), including for example if detecting sensor readings outside of acceptable ranges, significant changes in medication dispensing, medical questions or medical emergency notifications, by way of example.

Therein, the control module 102 allows for central user control for monitoring patient status, dispensing medication, and communicating with control systems external to the patient. As used herein, the term patient and user may be interchangeable. The patient is the individual receiving the medication and may be the person, user, controlling or interacting with the control module 102. Whereas, the patient could have a caretaker or attendant also assisting. For example, a patient may receive the medication but a caretaker or attendant may operate the touchscreen, keypad, mobile device, etc. in response to instructions from the patient. One example may be user interaction questions, such as asking how the patient is feeling, the caretaker or attendant could ask the question to the patient and then enter the information for the control module 102 or the patient can manually enter that information themselves.

As part of pain management, the analgesic dispensing unit 104 of FIG. 3 helps dispense medication. A primary concern of any pain management is proper dosage of medicine. Too little dosage results in unnecessary pain and too high of a dosage can be fatal. Similarly, as the present method and system provides for ambulatory pain management, concerns arise with proper handling and care of the medications.

In one embodiment, the analgesic is dispensed using a peripheral continuous nerve block catheter. As noted above, one exemplary embodiment may be a catheter described in the '351 Patent. FIG. 4 illustrates a side elevational view of a catheter assembly 200. The catheter assembly 200 is of a diameter which allows the assembly to be inserted through a typical needle assembly and into the body of the patient. According to one embodiment, the catheter assembly has a diameter range of twelve (12) to twenty-two (22) gauge. The catheter assembly 200 includes three portions: a central portion 204, a proximal portion 206 and a distal portion 202. The catheter assembly 200 is placed in the body of the patient with the distal portion 202 entering the body first.

The catheter assembly 200 includes an electrically conductive wire 216, which spans the length of the catheter assembly 200. At the central portion 204 of the catheter assembly 200, a sheath 214 covers the electrically conductive wire 216. The sheath 214 is formed from a thermoplastic or some other similar material in order to insulate an electrical charge that will be conducted through the wire 216. The sheath 214 defines a central bore 212 through which a liquid may pass freely. At the proximal portion 206 of the catheter assembly 200, the wire 216 is not covered by the sheath 214 and has a length that is shorter relative to central portion 204 of the catheter assembly 200. The wire 216 is left exposed so that an electrical charge can make contact with it, in order to conduct an electrical charge down its entire length. According to one embodiment, an electric stimulator as is known in the art, can make contact with the exposed portion of the wire 216 in order to provide the electrical charge.

The distal portion 202 of the catheter assembly 200 also has a length that is shorter relative to central portion 204 of the catheter assembly 200 and is not covered by the sheath 214. The electrically conductive wire 216 is left exposed at the distal portion 202 in order to allow the electrical charge to make contact with a target nerve (Not shown). Attached to the electrically conductive wire 216 at the distal portion 202 is an electrically conductive tip 218, which in one embodiment, is a rounded tip made of a material capable of conducting an electrical current. According to one embodiment, the electrically conductive tip 218 is a piece of rounded metal.

The catheter assembly 200 further includes an inflatable balloon 226 that is located toward the distal end of the catheter assembly 200, as shown in FIG. 4 in a deflated state. The inflatable balloon 226 can be expanded with either a gaseous substance or a liquid substance, such as saline or a local anesthetic. A gaseous or liquid substance is delivered to the inflatable balloon 226 through a balloon channel 220. The balloon channel 220 terminates within the inflatable balloon 226, allowing for the gaseous or liquid substance to exit the balloon channel 220 through a balloon channel opening 224. The balloon channel 220 extends from the opening 224 along the length of the central portion 204 and the proximal portion 206 of the catheter assembly 200. At the proximal end of the channel 220 is a balloon channel injection opening 222, where the gaseous or liquid substance can be injected into the channel 220. Once the gaseous or liquid substance is injected into the channel 220, the gaseous or liquid substance is delivered to the balloon 226 through the opening 224, allowing for the balloon 226 to expand to an inflated state. According to one embodiment, in its inflated state, the balloon 226 will have a diameter range of 0.1 to 3 cm.

Inflating the balloon 226 assists in catheter placement. Injection of liquid, such as for example liquid having microbubbles therein, significantly increases visibility of the needle placement next to a patient's nerve. By improving ease of installation, the peripheral continuous nerve block is accessible to more patients by reducing operational error in placing the catheter in the first instance.

The nerve block 200 further includes connectivity to analgesic dispensing unit (104 of FIG. 3) for dispensing analgesic solution via the central bore 212.

As part of dispensing analgesics, FIG. 5 illustrates one embodiment of the analgesic dispensing unit 104. The unit 104 includes an embodiment utilizing two containers, a first vial 260 includes a first liquid and a second vial 262 contains a second liquid. The vials engage a regulator 264 that combines and then dispenses the liquid to a catheter, such as 200 of FIG. 4.

In one embodiment, the first vial 260 includes an analgesic liquid for pain management, such as non-opioid analgesic, opioid(s), or other medicine, such as for example ropivacaine as one example but not limiting as the only type of analgesic as recognized by one skilled in the art.

The second vial 262 includes a dilution liquid, such as a saline solution, for subsequent mixture with the analgesic liquid. Changes in the mixture ratio changes the dispensed dosage of medication. Thereby, the patient does not require a large quantity of pain medication, but can utilize a smaller quantity with mixing. By way of example, the dilution can produce a dosage of ropivacaine between 0.2-0.5%, as one exemplary embodiment and not expressly limiting in types or dosage percentage for pain management as recognized by one skilled in the art.

The distribution of medication can then be adjusted not only by the flow rate but also by the mix rate between the two vials 260, 262.

In one embodiment, the regulator 264 receives a mix rate from the control module (102 of FIG. 1). Based on this mix rate, the regulator 264 pulls the proper amount of liquids from the vials 260 and 262 for dispensing through the catheter. As the control module (102 of FIG. 1) changes a mix rate based on user feedback or other parameters, this then changes the pull rate from the associated vials 260, 262.

Similarly, the regulator 264 may operate with the control module (102 of FIG. 1) to monitor liquid reserves. For example, one embodiment may include instructing the control module to notify the network controller 112 requesting a prescription refill.

In one embodiment, a prescription refill can include one or more replacement vials. Sensors or other electronics may detect not only remaining liquid amounts, but can detect vial removal. Therefore, the dispensing unit 104 can include functionality for monitoring vials of high dosage of analgesics. The vials can also be removed and new ones inserted using any suitable known engagement and/or refillable cartridge container technologies.

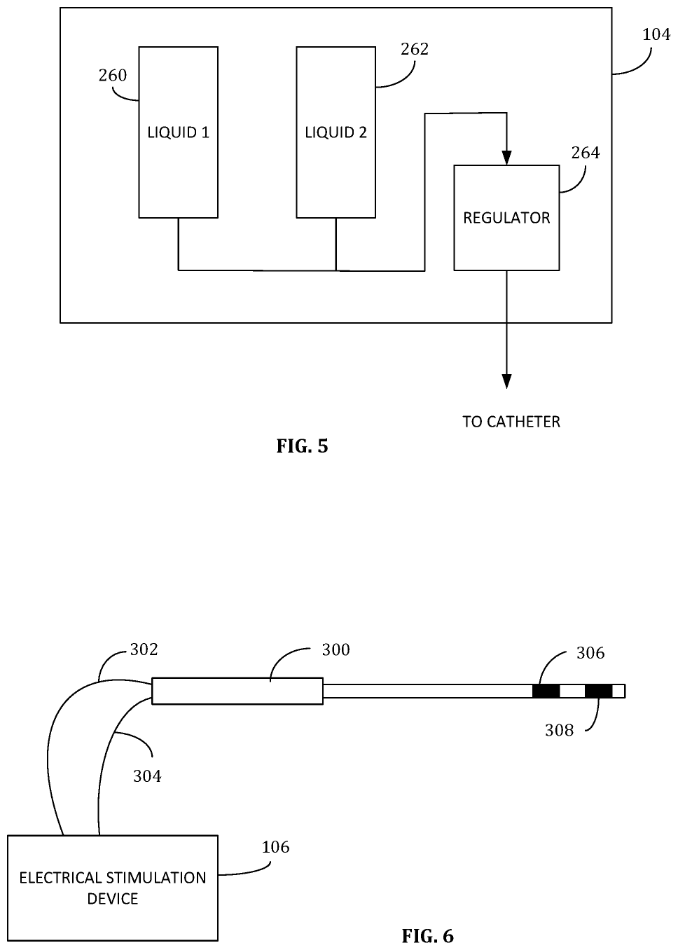

FIG. 6 illustrates one embodiment of an electrical stimulation device 300 for generating electrical stimulation against a nerve of the patient. The device 300 includes the controller 106 providing a positive lead 302 and negative lead 304 for supplying electrical current to the patient via conducting elements, such as cathode 306 and anode 308.

Any suitable neuromodulation device, as recognized by one skilled in the art, may be utilized herein. Neuromodulation controls may include electrical charges consistent with known neuromodulation therapy techniques, including for example described in U.S. Pat. No. 8,275,461.

In one embodiment, the neuromodulation may be generated using existing catheter and nerve blocks already engaged to the patient. For example, the nerve block of FIG. 4 includes the electrically conductive wire 216. In other embodiments, the neuromodulation may be conducted on the patient using a separate nerve block and/or electrical stimulation assembly.

The SAID provides for improvement over standard analgesic therapy by using the control module to switch pain management modalities. Thus, the patient can reduce dependence on analgesic medication, receiving neuromodulation. The controller 106, in response to an activation command from the control module (not shown in FIG. 6) then provides the electrical stimulation for pain management and therapeutic purposes. The amount of electrical discharge, including frequency, current, duration, etc., are subject to variations based on any number of factors, as recognized by one skilled in the art.

Therein, based on response to activation from the control module, the controller 106 can then subject that patient to pain management using the neuromodulation in place of the dispensing of analgesics.

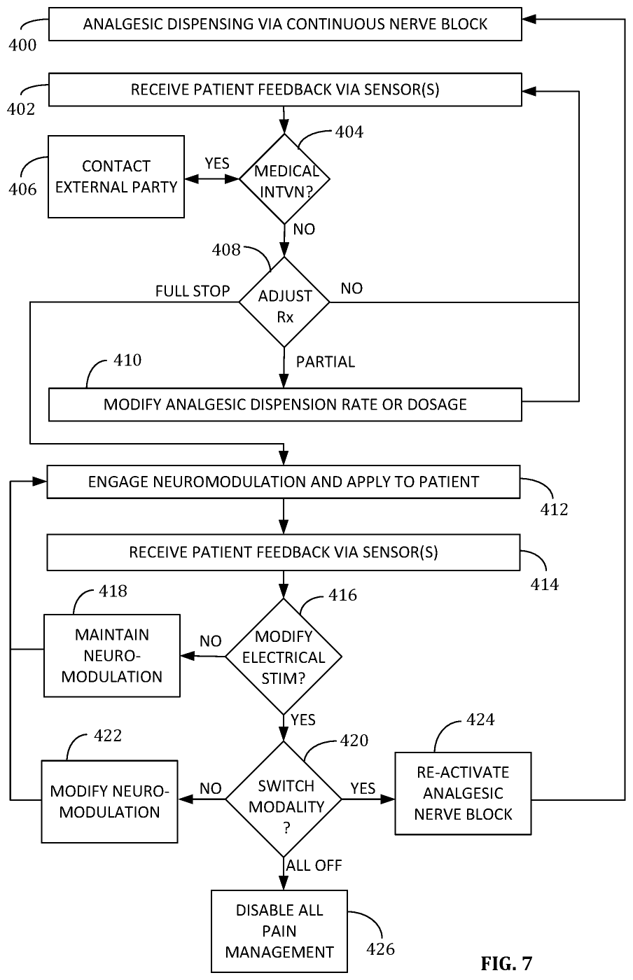

FIG. 7 illustrates a flowchart of the steps of one embodiment of a method for multi-modal pain management. The method can be performed using the SAID as described above.

The monitoring of patients as described herein can be performed in any suitable remote or distance-based environment. In one embodiment, the patient is discharged from the hospital and sent either home or to a secondary residential facility, with distance-based monitoring via the SAID. In another embodiment, the SAID can provide pain management while still in a hospital or medical care clinic. For example, the patient may be moved from a surgery wing to a recovery or a remote-monitoring wing, where the patient is able to receive medical attention if needed. But, using the SAID allows for nurses or other medical staff to not have to directly and in-person monitor the patient pain medicine, such as needing to enter the room every hour or so, taking up valuable hospital resources. Thus, the SAID as described herein can operate within a hospital or a non-hospital setting.

Step 400 is dispensing analgesic via the continuous peripheral nerve block catheter. As noted above, the dosage is adjustable using the dual liquid and the regulator of FIG. 5. While the patient is receiving the analgesic, step 402 is to receive patient feedback via the sensors. Based on this feedback, step 404 is a decision if medical intervention is required. If yes, step 406 is to contact an external party, such as a doctor, ambulance, nurse, family member, visiting nurse, etc. If medical intervention is not necessary, the analgesic dispensing is maintained.

In step 408, a second decision is if there is a need to adjust the medication. Medication adjustment can be based on any number of factors. One factor is in response to sensor data, such as sensor data indicating a greater degree of patient mobility, sensor data indicating a reduction in pain sensation, etc. Another factor may be time-based, such as reducing medication based on an initial 24 hour dosage periodic with period reductions over the next 24 hours or any other time period.

If there is no medication adjustment, the method reverts to step 402 to continue medication and monitor patient via sensor feedback. If there is a partial adjustment, the method proceeds to step 410 of modifying the rate or dosage. The modification may be in response to a user command, doctor command, preset reduction, or any other suitable technique. Thereupon modification, the method again reverts to step 402.

If there is a full stop, the method proceeds to step 412, whereby the control module engages and applies electrical stimulation to the patient. It is noted that in one embodiment, the transition between steps 408 and 412 may not be automatic, but can for example have a preset time delay, such as allowing the medication to wear off prior to electrical stimulation.

In another example, a patient may be subject to physical therapy or other mobility or post-operative requirements, where the delay between modalities allows for recovery. For example, if the patient had a knee replacement surgery, the analgesic may be stopped 30 minutes before a physical therapy session to insure proper sensory feedback by the patient. Then, during therapy or shortly thereafter, the patient may receive pain management via the neuromodulation, step 412.

In step 414, the SAID continues to receive patient feedback via the sensors. Based on this feedback, step 416 determines if the electrical stimulation should be modified. If no, step 418 is to maintain the neuromodulation and revert back to step 412. If yes, step 420 is another inquiry of whether to switch modalities. Here, the controller can switch back from the neuromodulation to dispensing the analgesic.

If the inquiry of step 420 is no, thus modifying the electrical stimulation but not switching modalities, step 422 is to modify the neuromodulation. Whereby, the method reverts again to step 412.

If the inquiry of step 420 is to switch modalities, step 424 is to re-activate the analgesic nerve block. Therein, the method reverts to step 400 beginning again on dispensing the medication. In the event all pain management is terminated, step 426 provides for disabling both modalities.

Therein, the SAID provides for multi-modal pain management using both medication and neuromodulation. The SAID engages these modalities for nerve-blocking technologies. The SAID utilizes a dual-analgesic dispensing system for more effectively monitoring the dispensing of medicine. Additionally, the utilization of sensors and feedback components enable ambulatory usage by the patient outside of the direct supervision of medical care, reducing or eliminating unnecessary post-operative hospitalizations.

Ambulatory continuous peripheral nerve blocks (CPNBs) are becoming increasingly popular to provide safe, high-quality postoperative analgesia for outpatient surgery.

For suitable patients, this allows for drastic reductions in healthcare expenditures for many high-cost surgical procedures (Orthopedics, etc.) by avoiding lengthy hospital stays principally for pain control. These can also reduce staff overhead associated with unnecessary hospital stays, and allow the patient to recover quicker in the comfort of his or her own home.

As patients presenting for outpatient surgery become increasingly aged and complex, technology available to treat post-operative pain must concurrently advance to meet this need. With this has grown a sub-specialty within Anesthesiology: Acute Pain Medicine.

The Acute Pain Medicine physician's aim for ambulatory surgical patients is to provide safe and effective post-operative analgesic modalities, with the same level of monitoring, communication and customization that would be afforded had the patient been treated as an inpatient.

While technique and skill of many clinicians placing continuous nerve blocks safely and effectively is reaching maturity, current pump technology used for a CPNB infusion is limited. Existing products used for Ambulatory Infusion Pumps (AIPs) consist of mainly two types: elastomeric devices and electrical pumps. Elastomeric AIPs (examples being the On-Q Pump, TeleFlex AutoFusor, and Baxter Pump) are simple elastic reservoirs filled with local anesthetic medication. A restrictor in the outflow tubing governs infusion rate; but as the volume decreases, so too does the flow rate. This results in a clumsy, inaccurate device with no physician control beyond its initial settings. Electrical pumps (the Ambit pump is perhaps the most well-recognized) provide improved accuracy in the volume delivered and allow for improved bolus functionality, but are not intended to be patient-manipulated. A large-volume bag of local anesthetic medication to be infused is stored in a ‘fanny pack’, which is heavy and bothersome to many patients. Any increase in discomfort to the patient or complication with the prior art technology reduces the likelihood of patient compliance, thus negating benefits and eventually increasing medical costs. Neither prior art technology is able to communicate directly with the physician as many ‘smart’ technologies today are.

Local anesthetics have a long track record of success in acute postoperative analgesia when appropriately infused through peripheral continuous nerve block catheters. Another modality being adapted for use in post-operative pain patients is electrical stimulation or peripheral neuromodulation. By providing an electrical current to peripheral nerves of specific frequency and amplitude, patients experience a decrease in sensed pain. Currently, peripheral neuromodulation leads must be surgically implanted near the target nerve and connected to an externalized pulse generator.

The Smart Ambulatory Analgesic Integration Device (SAID) is the ideal postoperative analgesia tool. Such a device would provide local anesthetic pump capability with the accuracy and programmable bolus functionality found in today's electric anesthetic infusion pumps, as well as an integrated peripheral neuromodulation impulse generator which utilizes electrical waveforms that can be utilized with an existing indwelling continuous peripheral nerve infusion catheter.

The SAID further differs from existing devices by adding the ability to communicate with the physician and patient directly, be easily adjustable by the practitioner remotely, continuously monitor several key patient data points, and provide an overall improved patient experience.

Smart, connected devices are booming in the personal and home electronics markets. There is a demand for the principles behind the ‘internet of things’ to extend to the medical market. The SAID will communicate with the physician via relatively inexpensive Bluetooth technology connected to the patient's smartphone. Where this is unavailable, a direct cellular connection could be created. Data conveyed via these connections would be sourced from not only the pump itself but also from patient-connected sensors, described below. Remotely, the physician could monitor pump function, total dose of local anesthetic administered, battery life, and be able to make adjustments in rate of infusion, interval and volume of boluses, concentration of local anesthetic infusion (described below), and remotely stop the infusion at a scheduled time or earlier should complications arise. Since the device itself will have only two physical buttons: an emergency on/off switch and a bolus (extra supplemental dose) button, all adjustments in local anesthetic prescription will be done from the physician end during initial setup and remotely during use. Because the device is always connected to the physician, never again will patients with these ambulatory CPNBs be ‘unreachable’ after discharge.

In addition to the SAID's ability to connect directly to the physician, so, too shall it be able to connect with the patient via a wireless module placed in direct contact with the patient and with sensors within the pump itself. The SAID is a small device, intended to be worn on the patient's operative limb, integrated into the surgical brace or sling. Within the device an accelerometer can provide information regarding the patient's overall activity level and movement of the limb itself. Low levels of movement can indicate immobility secondary to excessive pain or an overly dense nerve block affecting motor function of the limb. A camera within the device could be used to analyze the patient's facial expressions and be used as an objective measure of pain. The device could alternatively solicit patient pain scores as a pre-programmed interval. Information from sensors present on the patient's smartphone or wearable (ie. Fitbit®, Apple Watch®) can be used to further improve activity monitoring.

A wireless sensor can be connected to the patient's operative limb that can be used to sense a number of physiologic variables including temperature of the limb, reflectance-based tissue oximetry to measure oxygenation of tissue noninvasively, electrocardiography, heart rate, respiratory rate and electromyographic data. Any abnormality in the data sensed would be immediately communicated to the medical provider as an alert. Multimodal evaluation of the patient allows for contextual awareness of patient data through use of probabilistic graphical models and other machine learning techniques.

In a further embodiment, the SAID is usable with a recovery pillow or other recovery assistance device. It is recognized in a post-operative environment, rest is extremely important for the patient. But it is shown that compression and elevation of an affected limb can also aid recovery. Elevation can improve comfort and compression helps improve blood flow, as well as allowing the body to perform its natural recovery processes.

The SAID can be used with compression pillows, including compression instructions made available from the control module 102 (FIGS. 1 and 3). For example, a compression pillow can include connection to an air pump or other air flow regulator device. The control module 102 can include instructions to inflate or deflate a compression pillow, sleep, etc.

In one embodiment, a brace can be fitted around the limb. The brace can include one or more air bladders. As the bladder(s) is(are) filled with air, this places strategic compression on the limb. The regulation of the bladder(s) regulates compression for improving patient recovery.

As elevation is part of the recovery process, pillows or other support accessories can include techniques for elevating or lowering the limb. These height elevation instructions may be controlled by the control module 102.

One exemplary embodiment providing both compression and elevation is a splint and pillow combination as noted in U.S. Pat. No. 10,292,858. The adjustment of elevation and limb compression can thus be controlled by the control module as part improving patient recovery.

In a further embodiment, the control module 102 or the SAID itself can be fitted within or otherwise attached to a splint or a pillow. For example, a leg compression sleeve can include a pocket or attachment to hold the SAID.

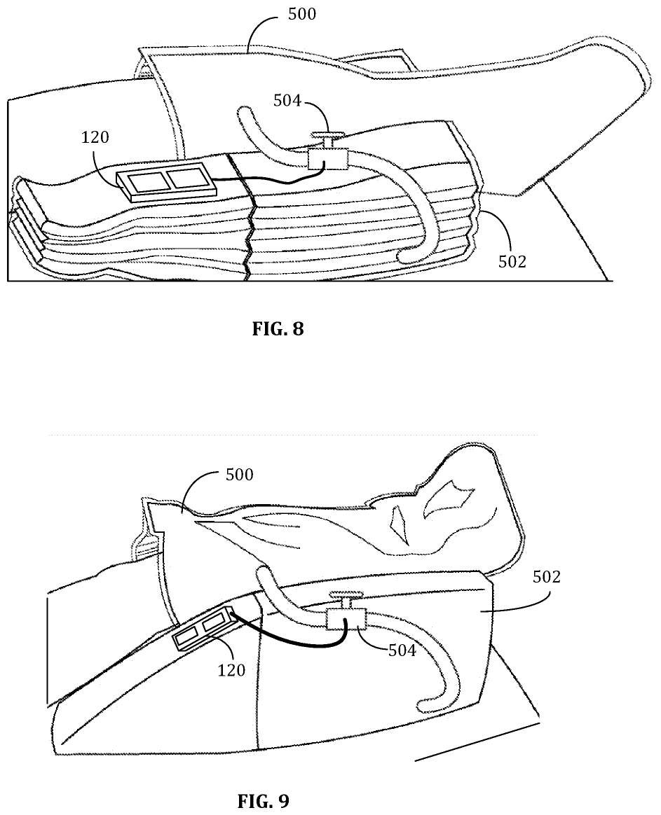

FIG. 8 illustrates one embodiment of a limb immobilization device usable with the SAID. The limb immobilization device, in this embodiment, includes a leg splint 500 and a pillow 502 combination. In this exemplary embodiment, the pillow 502 is a compressed pillow within an airtight sleeve. The splint 500 encases a limb, having one embodiment of a control module 120 disposed thereon or connected thereto.

The splint 500 and pillow 502 combination may operate similar to the techniques describes in U.S. Pat. No. 10,292,858, whereby inflated air bladder(s) in the splint 500 encase the limb, the pillow 502 housing in an airtight casing, the pillow compressed. This splint 500 and pillow 502 combination further includes a valve 504 controlling airflow therebetween. In this embodiment, the valve 504 may be an electronic valve controlled by a signal from the control module 120. In this embodiment, the control module 120 has a wire connection, but may also operate via wireless communication.

Consistent with limb compression and elevation, FIG. 9 illustrates a second embodiment where the valve 504 opens to release air from the splint 500, allowing the pillow 502 to expand, elevating the leg. In one embodiment, the splint 500 may include beads or other compression elements (not shown) such that as the splint compresses about the leg, the beads or other compression elements mold around the limb.

Herein, FIGS. 8-9 illustrate one exemplary embodiment of the control module 120 attached to and operative with a limb immobilization device. Attachment of the control module 120 may be via any suitable attachment device, such as a pocket, hooks-and-loop connectors, magnets, etc.

It is recognized the above embodiment of a limb mobilization device is exemplary and not expressly limiting. Further embodiments of limb immobilization devices can include a medical boot, a sling, compression socks or sleeves, standard elevation pillows, or any other suitable device or devices that immobilize a patient's limb as part of post-operative or post-trauma recovery.

The SAID provides a unique patient experience. Unlike large, heavy elastomeric devices with limited bolus functionality or current electric pumps connected to large volume bags of local anesthetic, the SAID differs in several ways. First, the device is small and easily wearable. Instead of a large, heavy reservoir of local anesthetic to carry around, the SAID administers medication via pre-filled infusion modules or cartridges. The SAID calculates and displays infusion time remaining on a given cartridge. The patient can then elect to carry only what medication is needed for a given period of time. This eliminates bulk.

The SAID that allows for adjustment of local anesthetic concentration remotely. As surgical pain decreases, the concentration of medication needed for analgesia too should decrease. In this case, the SAID would have two connected, changeable cartridges. One will contain a concentrated volume of local anesthetic and the other will contain normal saline. The two modules will be connected to the device with a safety mechanism to ensure the correct module is placed in the correct position-similar to a pin index safety system used in anesthetic gases. The advantage of this system is significant. By having parallel infusions, the concentration of the local anesthetic infusion that reaches the patient can be changed in real time by varying the relative rate of infusion of local anesthetic to normal saline. Should the SAID detect physiologic parameters consistent with a dense motor block post operatively, the concentration may be decreased remotely to allow for improved mobility while still providing appropriate analgesia. For some patients, an abrupt decrease in a local anesthetic infusion is not appropriate, and a slow wean of infusion volume alone may not allow spread of local anesthetic to all affected nerves. With a parallel infusion system such as this, the total infusion volume can be maintained while allowing a wean of local anesthetic concentration.

The concentration of local anesthetic that reaches the patient will be variable. Immediately post-operatively, patients may require a denser nerve block, only achieved by a more concentrated local anesthetic. As the patient's surgical site heals, pain should decrease proportionally. The SAID will take feedback cues from the patient's interaction with the pump and physiologic parameters. For example, if the patient does not indicate high pain levels, and requests infrequent bolus doses, the pump will aim to continually wean and minimize the concentration of local anesthetic as tolerated.

While acute surgical pain typically improves rapidly over the first 24-72 hours after a procedure, there is often persistent pain that can linger for weeks or more. Since it is inadvisable to continue a local anesthetic infusion for this period of time, most often the continuous nerve catheter is removed and the patient is treated with oral narcotic and non-narcotic pain medication. With the SAID's integrated neuromodulation functionality and impulse generator, electrical stimulation waveforms can be employed for a longer period of time, after the cessation of continuous local anesthetic infusion to decrease the need for oral narcotic medication.

The present method and device uses a plurality of known and existing techniques in a unique and new combination. The SAID includes unique functionality associated with existing PCNB technology and electrical stimulation. These two types of pain management modalities have always existed separate from each other, without combination because the prior nature of these modalities have been acute patient pain management and not a system for ambulatory care outside of a hospital setting.

The prior pain management modalities have been a single-source solution for pain management, as pain professionals, anesthesiologists advocate for one modality over the other. The typical implementation for pain modality selection is based on the care giver's experience with one modality over the other, as well as their comfort level with catheter insertion.

The use of a localized catheter typically means that if the insertion is off, the pain can be subjected to high degree of pain. Catheter placement is a precise procedure, misplacement causing analgesic to not contact the nerve in the correct dosage. Therefore, a medical professional having a fear of correct placement of one needle in a hospital setting teaches away from an obvious combination of multiple modalities available for an ambulatory setting. Wherein, the inclusion of the ultrasound based catheter improves accuracy of needle placement and electrical stimulation catheters provide automatic visual feedback curing clinician apprehension.

Moreover, the SAID uses newly available sensor technology for patient mobility and efficacy outside the hospital setting. Additionally, none of the prior solutions addressed mobile or ambulatory concerns for carrying liquid medication as solved by the analgesic dispensing unit in operation with the control module 102 herein.

FIGS. 1 through 7 are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, Applicant does not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments so fully reveals the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein.

What is claimed is:

1. A mobile pain management device for managing pain of a patient, the device comprising:
a peripherical continuous nerve block engageable with a first nerve of the patient;
an analgesic dispensing system having analgesic solution stored therein, the analgesic dispensing system connected to the peripheral continuous nerve block, the analgesic dispensing system including:
a first vial containing analgesic liquid;
a second vial containing dilution liquid; and
a regulator controlling a first flow rate of analgesic liquid and second flow rate of the dilution liquid;
a plurality of sensors sensor associated with the patient, the sensors detecting feedback from the patient;
a communication module in wireless communication with a network-based external system including transmission of at least a portion of the feedback from the patient detected by the sensors; and
a control module, in response to the wireless communication with the network-based external system, controlling the analgesic dispensing system to provide pain relieve to the patient via the peripheral continuous nerve block in response to at least a portion of the feedback from the patient.

2. The mobile pain management device of claim 1, wherein the network-based external system includes a care management system monitoring the at least a portion of the feedback from the patient, the device further comprising:
at least one output device displaying an update message notifying the patient about controls of the analgesic dispensing system based on the care management system.

3. The mobile pain management device of claim 2, wherein the output device includes a touchscreen for receiving data input regarding the patient, the device control module receiving user input regarding the patient directly via the touchscreen.

4. The mobile pain management device of claim 1, wherein the sensors includes at least one of: a heart rate monitor, a motion sensor, a camera, and a smart watch.

5. The mobile pain management device of claim 1 providing pain management for a patient, controlling pain with a first limb, the device further comprising:
a limb immobilization device immobilizing the first limb; and
an attachment device affixing the control module to the limb immobilization device.

6. The mobile pain management device of claim 1, the peripheral continuous nerve block including an inflatable balloon fillable with liquid for visibility during installation of the peripheral continuous nerve block within the patient.

7. A mobile pain management device for managing pain of a patient, the device comprising:
a peripherical continuous nerve block engageable with a first nerve of the patient;
an analgesic dispensing system having analgesic solution stored therein, the analgesic dispensing system connected to the peripheral continuous nerve block such that when active, the dispensing system provides at least a portion of the analgesic solution to the patient via the peripheral continuous nerve block;
an electrical stimulation device engageable with a second nerve of the patient such that when active, the electrical stimulation device provides an electrical stimulation to patient;
a plurality of sensors sensor associated with the patient, the sensors detecting feedback from the patient; and
a control module providing pain relieve to the patient through engagement of the peripheral continuous nerve block and the electrical stimulation device based on the feedback from the plurality of sensors.

8. The mobile pain management device of claim 7 further comprising:
a communication module in wireless communication with a network-based external system including transmission of at least a portion of the feedback from the patient detected by the sensors.

9. The mobile pain management device of claim 8, wherein the network-based external system includes a care management system monitoring the at least a portion of the feedback from the patient, the device further comprising:
at least one output device displaying an update message notifying the patient about the control module based on the care management system.

10. The mobile pain management device of claim 7, wherein the analgesic dispensing system includes:
a first vial containing analgesic liquid;
a second vial containing dilution liquid; and
a regulator controlling a first flow rate of analgesic liquid and second flow rate of the dilution liquid;
such that control module adjusts dispensing of analgesic solution via the regulator controlling the first flow rate and the second flow rate.

11. The mobile pain management device of claim 7, the control module, in response to executable instructions providing pain relieve to the patient by at least one of:
activating the analgesic dispensing system without activation of the electrical stimulation device and activating the electrical stimulation device without activating the analgesic dispensing system.

12. The mobile pain management device of claim 7, wherein the sensors includes at least one of: a heart rate monitor, a motion sensor, a camera, and a smart watch.

13. The mobile pain management device of claim 12 providing pain management for a patient, controlling pain with a first limb, the device further comprising:
a limb immobilization device immobilizing the first limb; and
an attachment device affixing the control module to the limb immobilization device.

14. A mobile pain management device for managing pain of a patient, the device comprising:
a peripherical continuous nerve block engageable with a first nerve of the patient;
an analgesic dispensing system having analgesic solution stored therein, the analgesic dispensing system connected to the peripheral continuous nerve block such that when active, the dispensing system provides at least a portion of the analgesic solution to the patient via the peripheral continuous nerve block, the analgesic dispensing system includes:
a first vial containing analgesic liquid;
a second vial containing dilution liquid; and
a regulator controlling a first flow rate of analgesic liquid and second flow rate of the dilution liquid;
such that control module adjusts dispensing of analgesic solution via the regulator controlling the first flow rate and the second flow rate.
an electrical stimulation device engageable with a second nerve of the patient such that when active, the electrical stimulation device provides an electrical stimulation to patient;
a plurality of sensors sensor associated with the patient, the sensors detecting feedback from the patient;
an input device operative to receive feedback from the patient;
a communication module in wireless communication with a network-based external system including transmission of at least a portion of the feedback from the patient detected by the sensors; and
a control module, in response to the wireless communication with the network-based external system, providing pain relieve to the patient in response to at least a portion of the feedback from the patient, providing the pain relieve via at least one of the peripheral continuous nerve block and the electrical stimulation device.

15. The mobile pain management device of claim 14, wherein the network-based external system includes a care management system monitoring the at least a portion of the feedback from the patient, the device further comprising:
at least one output device displaying an update message notifying the patient about controls of the analgesic dispensing system based on the care management system.

16. The mobile pain management device of claim 15, wherein the input device includes a touchscreen for receiving data input regarding the patient, the control module receiving user input regarding the patient directly via the touchscreen.

17. The mobile pain management device of claim 14, wherein the sensors includes at least one of: a heart rate monitor, a motion sensor, a camera, and a smart watch.

18. The mobile pain management device of claim 14 providing pain management for a patient, controlling pain with a first limb, the device further comprising:
a limb immobilization device immobilizing the first limb; and
an attachment device affixing the control module to the limb immobilization device.

19. The mobile pain management device of claim 14, the peripheral continuous nerve block including an inflatable balloon fillable with liquid for visibility during installation of the peripheral continuous nerve block within the patient.

20. The mobile pain management device of claim 14, wherein the first nerve and the second nerve are the same nerve within the patient.
